(19) United States
(12) Patent Application Publication
(10) Pub. No.: US 2006/0034778 A1
(43) Pub. Date: Feb. 16, 2006

(54) Packaged personal care compositions

(76) Inventors: Yoshinhisa Kitano, Haga-gun (JP); Atsushi Yatoh, Haga-gun (JP)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(21) Appl. No.: 10/529,027

(22) PCT Filed: Sep. 19, 2003
(86) PCT No.: PCT/EP03/10506

§ 371 Date: Mar. 21, 2005

(30) Foreign Application Priority Data
Sep. 27, 2002 (GB) 02225001

(51) Int. Cl. A61K 8/73; A61K 8/00

(52) U.S. Cl. 424/47; 424/70.17

(57) ABSTRACT

A packaged mousse-forming, personal care composition, comprising an aluminium based container with an inner surface coating of a thermosetting resin, wherein the composition comprises an aqueous base and a propellant, wherein the aqueous base comprises 0.16% or less by weight of chloride ions and has a pH of from 5.0 to 8.0.

### FIELD OF THE INVENTION

The present invention relates to specific mousse-forming personal care compositions, which are packaged in aluminium cans having an inner coating of cured polyamideimide resin. This combination allows storage under extreme conditions without corrosion to the container.

### BACKGROUND

Mousses are a particularly convenient and pleasant-to-use product form for personal care formulations. Consumers appreciate the ease of dispensing and application of a mousse. The product is generally applied to the user's hand, where it forms a creamy foam which can be easily worked through the hair or over the body.

Such mousses have found widespread use in the context of hair styling products but more recently, rinse-off surfactant-based cleansing shampoos in a mousse product form have also been disclosed. Such aerosol shampoos can be worked through the hair without getting into the eyes, which is of particular advantage in the context of formulations based primarily on cleansing surfactants which can sometimes be harsh and irritating to the eyes.

Aerosol containers must be pressurisable and are commonly metallic, traditionally tin plated. A well known problem in the field of aerosols is that of reaction of the metallic container with its contents. This leads to corrosion of the container as well as deterioration of the product contained therein. Corrosion is manifest as, for example, unsightly deposits in the composition and loss of propellant. When corrosion is severe enough it may ultimately cause leaking of the can in addition to contamination of the product inside. For example, oxidation can transform formulation components, such as those constituting the fragrance, with consequential adverse effects on odour. Thus, stringent corrosion control is needed for personal care products.

The problem of corrosion is particularly apparent when the can is used to contain water-based formulations and is further exacerbated by the presence of electrolytes such as salts and surfactants. Thus, corrosion is a particular problem for formulations such as shampoos which typically contain high levels of sodium chloride as well as surfactant systems.

In an attempt to overcome this problem, aluminium cans have been employed. However, aluminium cans can also be susceptible to corrosion. A further technique is to introduce a highly resistant coating on the inner surface of the container. However, to date, we have been unable to find a satisfactory corrosion resistant container and aerosol formulation system, in which the formulation is water based and contains sufficiently high levels of electrolyte to perform its function, which meets the high quality demands and storage life of today's personal product market.

We have now found that corrosion can be inhibited by use of an aqueous personal care composition with defined chloride ion level and pH characteristics in a container, which is aluminium and coated with a cured polyamideimide resin.

### PRIOR ART

U.S. Pat. No. 5,750,223 (Tada and Hayashi) discloses an inner surface coated container, exhibiting resistance against corrosion and adsorption of the contents. The coating material is a thermosetting, cured polyamideimide of defined absorbency, which is applied to the inner surface of a metallic aerosol can. The polyamideimide coating is preferably formed from a polyamideimide resin and a curing agent comprising an epoxy resin. A wide range of contents suitable for use with such a container, such as foods, hair dyes and similar chemicals is disclosed.

WO 99/32070 (Unilever) discloses a mousse-forming shampoo composition having improved conditioning performance. The composition comprises surfactant, emulsified conditioning agent, deposition polymer and propellant, for use in a pressurisable container. The problem of delivering higher levels of conditioning to hair from an aerosol shampoo is addressed.

### DEFINITION OF THE INVENTION

According to a first aspect of the invention, there is provided a mousse-forming personal care composition packaged in an aluminium container, wherein the composition comprises:
(a) from 80 to 98% by weight of an aqueous base comprising at least 30%, preferably at least 40%, most preferably at least 50% by weight of water and;
(b) from 2 to 20% by weight of a propellant,
   wherein the aqueous base (a) comprises 0.16% or less by weight of chloride ions and wherein the pH of the aqueous base (a) is from 5.0 to 8.0, and wherein the aluminium container has an inner surface coating of a thermosetting resin.

According to a second aspect of the invention, there is provided a process for the preparation of a packaged mousse forming composition of the invention, comprising the steps of:
(i) charging the aluminium container with the aqueous base,
(ii) sealing the container, and
(iii) adding the propellant through a valve.

### DETAILED DESCRIPTION OF THE INVENTION

The mousse-forming personal care composition of the invention is packaged in an aluminium container, which has a resistant inner surface coating of a thermosetting resin, preferably a cured polyamideimide resin.

The mousse-forming personal care composition of the invention comprises an aqueous base (a) and a propellant (b). The term “aqueous base” will be used to refer to the liquid component of the personal care composition other than the propellant. The term “mousse”, as used herein, is the same as foam, and refers to the dispensed product unless otherwise specified.

Aqueous Base (a)

The aqueous base (a) is present in an amount of from 80% to 98%, preferably 83% to 91% by weight of the mousse forming personal care composition.

The aqueous base comprises water, which is present in an amount of from at least 30%, preferably at least 40%, most preferably at least 50%. Typically, the aqueous base comprises 50% to 99%, preferably 50% to 90%, most preferably 55% to 80% by weight of the aqueous base of water.

The aqueous base (a) preferably has a viscosity of than 500 mPas or less (as measured with a cone and plate rheometer at 21 s⁻¹ and 25° C.).

Weight of Chloride Ions

The aqueous base comprises not more than 0.16% by weight of chloride ions, preferably in an amount of from 0 to 0.15%.

Sources of chloride ions are dependent upon the type of personal care composition. For example, sodium chloride is commonly used as a thickener or viscosity modifier in shampoos. Chloride ions are also a common impurity in some surfactants or form the counter ion in some ionic compounds.

pH of the Aqueous Base

It is an essential feature of the invention that the aqueous base has a pH (as measured with a glass electrode at 25° C.) in the range of from 5.0 to 8.0, more preferably from 5.5 to 7.0. For the sake of clarity, the pH is that of the aqueous base.

The stability benefits of the invention have been found to be especially marked under neutral pH conditions.

Aqueous personal care compositions according to the invention preferably comprise a pH adjusting agent. This agent can be any suitable material, which is capable of changing the pH of the composition. Such materials are well known to those skilled in the art. The pH adjusting agent is preferably selected from citric acid, arginine and mixtures thereof.

Shampoo Compositions

The corrosion-inhibiting conditions of the present invention are particularly useful where the aqueous base is a shampoo composition. By shampoo composition is meant a rinse off cleansing composition such as hair shampoo or shower-gel.

Sources of chloride ions in shampoos (that is shampoos which are not in accordance with the invention) are commonly as impurities in surfactants or from sodium chloride, which is used as a thickener or viscosity modifier.

According to the invention, shampoo compositions must have a pH and a chloride ion concentration as described above.

Shampoo compositions in accordance with the invention preferably comprise one or more cleansing surfactants which are cosmetically acceptable and suitable for topical application to the hair. Further surfactants may be present as emulsifiers for any hydrophobic compounds present in the composition.

Suitable cleansing surfactants, are selected from anionic, amphoteric and zwitterionic surfactants, and mixtures thereof. The cleansing surfactant may be the same surfactant as the emulsifier, or may be different. Preferably, low salt and more preferably salt free surfactants are used. It is particularly preferred to use low salt and salt free betaines, where betaines are employed. As a consequence of the process of manufacture, the amphoteric surfactants, betaines, usually contain sodium chloride. By low salt surfactant is meant a surfactant in which the weight ratio of surfactant to sodium chloride is preferably 30:1 or more, more preferably 300:1 or more and most preferably 600:1 or more. A suitable salt free betaine is Lebon 2000HG, supplied by Sanyo Chemical Industries, Japan. For the sake of clarity, the term betaine as used here does not include the meaning, trimethylglycine.

Anionic Cleansing Surfactant

Shampoo compositions suitable for use in the invention will typically comprise one or more anionic cleansing surfactants which are cosmetically acceptable and suitable for topical application to the hair.

Examples of suitable anionic cleansing surfactants are the alkyl sulphates, alkyl ether sulphates, alkaryl sulphonates, alkanoyl isethionates, alkyl succinates, alkyl sulphosuccinates, N-alkyl sarcosinates, alkyl phosphates, alkyl ether phosphates, alkyl ether carboxylates, alkyl ester carboxylates, and alpha-olefin sulphonates, especially their sodium, magnesium, ammonium and mono-, di- and triethanolamine salts. The alkyl and acyl groups generally contain from 8 to 18 carbon atoms and may be unsaturated. The alkyl ether sulphates, alkyl ether phosphates and alkyl ether carboxylates may contain from 1 to 10 ethylene oxide or propylene oxide units per molecule.

Typical anionic cleansing surfactants for use in the invention include sodium oleyl sulpho succinate, ammonium lauryl sulphosuccinate, ammonium lauryl sulphate, sodium cocoyl isethionate, sodium lauryl isethionate and sodium N-lauryl sarcosinate. The most preferred anionic surfactants are sodium lauryl sulphate, sodium lauryl ether sulphate(n)EO, (where n is from 1 to 3), ammonium lauryl sulphate and ammonium lauryl ether sulphate(n)EO, (where n is from 1 to 3).

The total weight of anionic cleansing surfactant in shampoo compositions, which are suitable for use in the invention, is generally from 5 to 30, preferably from 6 to 20, more preferably from 8 to 16 percent by weight of the composition.

Co-Surfactant

The shampoo composition can optionally include co-surfactants, preferably an amphoteric or zwitterionic surfactant, which can be included in an amount ranging from 0 to about 8, preferably from 1 to 4 percent by weight of the composition. These co-surfactants are preferably low salt and more preferably salt free.

Examples of amphoteric and zwitterionic surfactants include, alkyl betaines, alkyl amidopropyl betaines, alkyl sulphobetaines (sultaines), alkyl glycinates, alkyl carboxyglycinates, alkyl amphopropionates, alkylamphoglycinates, alkyl amidopropyl hydroxysultaines, acyl taurates and acyl glutamates, wherein the alkyl and acyl groups have from 8 to 19 carbon atoms. Typical amphoteric and zwitterionic surfactants for use in shampoos of the invention include lauryl amine oxide, cocodimethyl sulphopropyl betaine and preferably lauryl betaine, cocamidopropyl betaine and sodium cocamphopropionate.

Another preferred co-surfactant is a nonionic surfactant, which can be included in an amount ranging from 0 to 8, preferably from 2 to 5 percent, by weight of the composition.

For example, representative nonionic surfactants that can be included in shampoo compositions of the invention include condensation products of aliphatic (C₈-C₁₈) primary or secondary linear or branched chain alcohols or phenols with alkylene oxides, usually ethylene oxide and generally having from 6 to 30 ethylene oxide groups.

Further nonionic surfactants which can be included in shampoo compositions of the invention are the alkyl polyglycosides (APGs). Typically, the APG is one which comprises an alkyl group connected (optionally via a bridging group) to a block of one or more glycosyl groups. Preferred APGs are defined by the following formula:
RO−(G)ₙ

wherein R is a branched or straight chain C₅ to C₂₀ alkylor alkenyl group, G is a saccharide group and n is from 1 to 10.

Other sugar-derived nonionic surfactants which can be included in shampoo compositions of the invention include the C₁₀-C₁₈ N-alkyl (C₁-C₆) polyhydroxy fatty acid amides, such as the C₁₂-C₁₈ N-methyl glucamides, as described for example in WO 92/06154 and U.S. Pat. No. 5,194,639, and the N-alkoxy polyhydroxy fatty acid amides, such as C₁₀-C₁₈ N-(3-methoxypropyl)glucamide.

Cationic Surfactant

The shampoo composition can also optionally include one or more cationic co-surfactants included in an amount ranging from 0.01 to 10, more preferably from 0.05 to 5, most preferably from 0.05 to 2 percent by weight of the composition. Cationic surfactants useful in compositions of the invention contain amino or quaternary ammonium hydrophilic moieties which are positively charged when dissolved in the aqueous composition of the present invention.

Cationic Polymer

The shampoo composition can optionally include cationic polymer(s). These may be homopolymers or be formed from two or more types of monomers. The molecular weight of the polymer will generally be between 5,000 and 10,000,000, typically at least 10,000 and preferably from 100,000 to about 2,000,000 Da. The polymers will have cationic nitrogen containing groups such as quaternary ammonium or protonated amino groups, or a mixture thereof.

Suitable cationic nitrogen polymers are described in the CTFA Cosmetic Ingredient Directory, 3rd edition

The cationic polymers can comprise mixtures of monomer units derived from amine- and/or quaternary ammonium-substituted monomer and/or compatible spacer monomers.

Suitable cationic polymers include, copolymers of 1-vinyl-2-pyrrolidine and 1-vinyl-3-methyl-imidazolium salts (CTFA name Polyquaternium-16); copolymers of 1-vinyl-2-pyrrolidine and dimethylaminoethyl methacrylate, (CTFA name Polyquaternium-11); cationic diallyl quaternary ammonium-containing polymers in particular CTFA Polyquaternium 6 and Polyquaternium 7, Polyquaternium 47, mineral acid salts of amino-alkyl esters of homo-and co-polymers of unsaturated carboxylic acids as described in U.S. Pat. No. 4,009,256 and cationic polyacrylamides (as described in WO95/22311).

Cationic polysaccharide polymers suitable for use in aqueous base compositions of the invention include those with an anhydroglucose residual group, such as a starch or cellulose. Cationic cellulose is available from Amerchol Corp. (Edison, N.J., USA) in their Polymer JR (trade mark) and LR (trade mark) series of polymers, as salts of hydroxyethyl cellulose reacted with trimethyl ammonium substituted epoxide, referred to in the industry (CTFA) as Polyquaternium 10. Another type of cationic cellulose includes the polymeric quaternary ammonium salts of hydroxyethyl cellulose reacted with lauryl dimethyl ammonium-substituted epoxide, referred to in the industry (CTFA) as Polyquaternium 24. These materials are available from Amerchol Corp. (Edison, N.J., USA) under the tradename Polymer LM-200.

Other suitable cationic polysaccharide polymers include quaternary nitrogen-containing cellulose ethers (e.g. as described in U.S. Pat. No. 3,962,418), and copolymers of etherified cellulose and starch (e.g. as described in U.S. Pat. No. 3,958,581).

A particularly suitable type of cationic polysaccharide polymer that can be used is a cationic guar gum derivative, such as guar hydroxypropyltrimonium chloride (commercially available from Rhodia in their JAGUAR trademark series). Particularly preferred cationic polymers are JAGUAR C13S, JAGUAR C14, JAGUAR C15, JAGUAR C17 and JAGUAR C16 Jaguar CHT and JAGUAR C162. A further preferred cationic polymer is Polyquaternium 47, commercially available from Nalco Chemical Company, USA.

The cationic polymer will generally be present in aqueous base compositions of the invention at levels of from 0.01 to 5, preferably from 0.05 to 1, more preferably from 0.08 to 0.5 percent by weight of the composition.

Propellant (b)

Compositions of the invention contain an aerosol propellant (b). This agent is responsible for expelling the other materials from the container and forming the mousse character.

The propellant gas can be any liquefiable gas conventionally used for aerosol containers. Examples of suitable propellants include dimethyl ether, propane, n-butane and isobutane, used singly or admixed. A preferred propellant of the present invention is an admixture of propane, n-butane and isobutane, such as BPAP 40, BPAP 48 and BPAP 70, all supplied by British Petroleum. A further preferred propellant is LPG (Liquefied Petroleum Gas). Other examples of propellants are nitrogen, carbon dioxide, compressed air and fluorohydrocarbons such as the material sold by Du Pont under the trade name DYMEL 152a.

For mousses of the invention, the level of propellant is from 2% to 20%, preferably from 3% to 15%, optimally from 4% to 10% by weight of the personal care composition.

Optional Ingredients

Compositions according to the present invention may contain any other ingredient normally used in personal care compositions, and depending on the intended use of the composition. These other ingredients may include bacteriostats (such as triclosan) for deodorants, perspiration inhibitors (such as aluminium or zirconium salts) for antiperspirants, conditioning agents (such as emollients, lubricants and moisturisers), for skin or hair conditioning products such as post-wash hair conditioners, hair or body conditioning shampoos and shaving foams, colouring agents, antifoam agents, antioxidants, fragrances, antimicrobials, solvents for components such as hair styling polymers, and sunscreens. Each of these ingredients will be present in an amount effective to accomplish its purpose, generally at a level of up to 2%, preferably up to 1%, by weight of the total composition.

Examples of preferred optional ingredients in compositions of this invention are adjuvants suitable for hair care. These may include hair styling resins, colouring agents, proteins, amino acids, moisturising agents, fragrances, antimicrobial agents, sunscreens and hair fibre benefit agents (e.g. ceramides and fatty acids).

Mixtures of any of the above active ingredients may also be used.

Aluminium Based Container

Containers for use in the invention are any containers suitable for use as aerosol containers, which comprise aluminium. Preferably, the container is pure aluminium, or aluminium alloy, comprising at least 90 wt % of aluminium. Suitable containers include those described in U.S. Pat. No. 5,750,223.

Thermosetting Resin

The thermosetting resin of the invention can be any suitable resistant thermosetting resin.

The thermosetting resin preferably has a low permeability to protons.

The thermosetting resin preferably comprises chemical groups selected from the group consisting of amino groups, carboxyl groups, and mixtures thereof.

The thermosetting resin is preferably free of polyvinylorganosols such as those made from polyvinylchloride (PVC) and epoxyphenolic resins.

In a highly preferred embodiment, the thermosetting resin is a cured polyamideimide resin.

The cured polyamideimide resin is preferably formed of a composition comprising a polyamideimide resin and a curing agent. The curing agent preferably consists essentially of an epoxy resin.

A suitable cured polyamideimide coating material for use in the invention is obtained by mixing a solution of a polyamideimide resin and a solution of a curing agent (preferably an epoxy resin). Particularly suitable organic solvents for obtaining these solutions are those normally used in the production of can coatings, such as N-methyl-2-pyrrolidone, xylene, methyl ether ketone, etc and mixtures thereof. Further agents may be added to the polyamideimide/epoxy resin mixture, including reforming resins, lubricating agents, viscosity-decreasing agents, stabilisers, anti-oxidising agents, crosslinking agents and curing catalysts. The cured polyamideimide coating is formed by applying said mixture of polyamideimide resin and curing agent onto the inner surface of an aluminium aerosol container and then curing at a predetermined temperature. A preferred method of applying the coating is by spray drying.

Suitable cured polyamideimide resins, their synthesis and methods of applying such to aerosol containers are described in U.S. Pat. No. 5,750,223.

Method of Packing

Compositions of the invention are typically prepared by first preparing the aqueous base (a) according to techniques well known to those skilled in the art. The inner surface coated aluminium container is then charged with the aqueous base (a) and sealed according to conventional techniques before the propellant (b) is added. The propellant is preferably added through a valve. The preferred means of sealing the containers is by crimp sealing.

### EXAMPLES

The invention will now be further illustrated by the following, non-limiting Examples, in which parts and percentages are by weight of the total composition (aqueous base plus propellant).

TABLE A

| Chemical | Trade Name |
|---|---|
| Sodium Lauryl ether Sulphate (SLES) (27% active) | *EMAL 227 HP |
| Cocoamidopropyl Betaine (CAPB) (30% active) | *Amphitol 55AB |
| Low salt Cocamidopropyl Betaine (30% active) | *Lebon 2000HG |
| Sodium Cocoyl Isethionate | *JORDAPON CI |
| Quaternised guar | *JAGUAR C-14-S |
| Cationic polymer | *MERQUAT 2001 |
| Dimethiconol | SILICONE |
| | CF2216 |

*Trade Mark

### Example 1

An aluminium container with a cured polyamideimide coating on its inner surface was charged with a shampoo base having the composition given in Table 1 to make the packaged composition of Example 1. Low salt Cocamidopropyl Betaine was used to minimise the chloride ion concentration. The chloride ion concentration was 0.15 wt % by weight of the total composition (shampoo base (a) +propellant (b)), and the pH of the shampoo base was 6.0. The aluminium container was then sealed and the propellant added (LPG, 0.37 MPa at a level of 5 wt % by total weight of the shampoo base+propellant).

TABLE 1

| | Weight %, by total |
|---|---|
| | weight of (a) + (b) |
| Component | Example 1 |
| Aqueous base (a) | |
| SLES | 10.0 |
| low salt CAPB | 6.0 |
| Sodium Cocoyl Isethionate | 2.0 |
| Disodium EDTA | 0.1 |
| Quaternised guar | 0.25 |
| Cationic polymer | 0.1 |
| Dimethiconol | 1.0 |
| Citric Acid | 0.07 |
| Ethanol (95 vol %) | 7.5 |
| Sodium Benzoate | 0.3 |
| Perfume | 0.4 |
| De-ionised water + minors | to 95.0 |
| Propellant (b) | |
| LPG (0.37 Mpa) | 5.0 |
| TOTAL | 100 |
| pH | 6.0 |
| Chloride ion concentration | 0.15 |
| (wt % of shampoo base + propellant) | |

* Trademark

### Comparative Examples A, B and C

Aluminium containers with an inner cured polyamideimide coating were charged with shampoo bases having the compositions given in Table 2 to make Comparative Examples A, B and C. Non-salt free Cocamidopropyl Betaine was used and the concentration of SLS was varied to alter the chloride ion concentration of the compositions. The pH was adjusted to 6.0 for each example. Comparative Examples A, B and C are not according to the invention because they have chloride ion concentrations of more than 0.16 wt %. The aluminium container was then sealed and the propellant was added (LPG, 0.37 MPa at a level of 5 wt % by total weight of the shampoo base+propellant).

TABLE 2

| | Weight %, by total weight | | |
|---|---|---|---|
| | of (a) + (b) | | |
| Component | A | B | C |
| Aqueous base (a) | | | |
| SLES | 10.0 | 16.2 | 12.0 |
| Non-salt free CAPB | 2.0 | 2.0 | 2.0 |
| Sodium Cocoyl Isethionate | 2.0 | 2.0 | 2.0 |
| Disodium EDTA | 0.1 | 0.1 | 0.1 |
| Quaternised guar | 0.25 | 0.25 | 0.25 |
| Cationic polymer | 0.1 | 0.1 | 0.1 |
| Dimethiconol | 1.0 | 1.0 | 1.0 |
| Citric Acid | 0.07 | 0.07 | 0.07 |
| Ethanol | 7.5 | 7.5 | 7.5 |
| Sodium Benzoate | 0.3 | 0.3 | 0.3 |
| Perfume | 0.4 | 0.4 | 0.4 |
| Deionized water + minors | to 95.0 | to 95.0 | to 95.0 |
| Propellant (b) | | | |
| LPG (0.37 MPa) | 5.0 | 5.0 | 5.0 |
| TOTAL | 100 | 100 | 100 |
| PH | 6.0 | 6.0 | 6.0 |
| Chloride ion concentration | 0.20 | 0.27 | 0.20 |
| (wt % of shampoo base + | | | |
| propellant) | | | |

* Trademark

### Comparative Examples D and E

Aluminium containers with an inner cured polyamideimide coating were charged with shampoo bases having the compositions given in Table 3, to make Comparative Examples D and E. The citric acid and arginine contents were selected to alter the pH to a value not according to the invention. The aluminium container was then sealed and the propellant added (LPG, 0.37 MPa at a level of 5 wt % by total weight of the shampoo base+propellant).

TABLE 3

Weight %, by total
weight of (a) + (b)

| Component | D | E |
|---|---|---|
| Aqueous base (a) | | |
| SLES | 10.0 | 10.0 |
| Low salt CAPB | 6.0 | 6.0 |
| Sodium Cocoyl Isethionate | 2.0 | 2.0 |
| Disodium EDTA | 0.1 | 0.1 |
| Quaternised guar | 0.25 | 0.25 |
| Cationic polymer | 0.1 | 0.1 |
| Dimethiconol | 1.0 | 1.0 |
| Arginine | — | 0.5 |
| Citric Acid | 0.3 | 0.07 |
| Ethanol | 7.5 | 7.5 |
| Sodium Benzoate | 0.3 | 0.3 |
| Perfume | 0.4 | 0.4 |
| Deionized water + minors | to 95.0 | to 95.0 |
| Propellant (b) | | |
| LPG (0.37 MPa) | 5.0 | 5.0 |
| TOTAL | 100 | 100 |
| pH | 3.5 | 9.0 |
| Chloride ion concentration | 0.0015 | 0.0015 |
| (wt % of shampoo base + propellant) | | |

* Trademark

### Comparative Example F

An aluminium container with an inner coating of a thermosetting polyvinylorganosol, Micoflex (trademark), was charged with the shampoo base given in Table 1 above to form Comparative Example F. The aluminium container was then sealed and the propellant added (LPG, 0.37 MPa at a level of 5 wt % by total weight of the shampoo base+propellant).

### Example 5

### Evaluation of Storage Stability

The storage stability of Example 1 and Comparative Examples A to F were assessed by storing at 50° C. for 6 months. Stability was assessed by the appearance of visible effects of corrosion on the can, specifically the formation of pin holes (i.e. leakage of the inner contents of the can) and blisters (i.e. corrosion on the inner surface of the container).

TABLE 5

| | Example | | | | | | |
|---|---|---|---|---|---|---|---|
| | 1 | A | B | C | D | E | F |
| Coating | cured poly- | cured poly- | cured poly- | cured poly- | cured poly- | cured poly- | *Micoflex |
| | amideimide | amideimide | amideimide | amideimide | amideimide | amideimide | |
| pH | 6.0 | 6.0 | 6.0 | 6.0 | 3.5 | 9.0 | 6.0 |
| Chloride ion | 0.15 | 0.20 | 0.27 | 0.20 | 0.15 | 0.15 | 0.15 |
| concentration | | | | | | | |

Stability at 50° C.

| | | | | | | | |
|---|---|---|---|---|---|---|---|
| Time to | — | 5 months | 1 month | 3 months | 2 months | — | — |
| formation of | | | | | | | |
| blister | | | | | | | |
| Time to | — | — | 5 months | — | — | 3 months | 4 months |
| formation of | | | | | | | |
| pin hole | | | | | | | |

*Trademark

It will be seen that storage stability was dramatically improved by use of the composition in accordance with the invention in aluminium containers with a cured polyamideimide inner coating.

1. A mousse-forming personal care composition packaged in an aluminium container, wherein the composition comprises:
(a) from 80 to 98% by weight of an aqueous base comprising at least 30%, preferably at least 40%, most preferably at least 50% by weight of water and;
(b) from 2 to 20% by weight of a propellant,
wherein the aqueous base (a) comprises 0.16% or less by weight of chloride ions and wherein the pH of the aqueous base (a) is from 5.0 to 8.0, and wherein the aluminium container has an inner surface coating of a cured thermosetting resin, wherein the thermosetting resin is a cured polyamideimide resin.

2. A packaged composition as claimed in claim 1, wherein the aqueous base (a) is a shampoo base comprising a cleansing surfactant.

3. A packaged composition as claimed in claim 2, wherein the cleansing surfactant is present in an amount of from 5 to 40 wt % by weight of the total composition.

4. A packaged composition as claimed in claim 2, wherein the weight ratio of cleansing surfactant to sodium chloride is 30:1 or more, preferably 300:1 or more and more preferably 600:1 or more.

5. A packaged composition as claimed in claim 1, which further comprises a pH adjusting agent.

6. A packaged composition as claimed in claim 5, wherein the pH adjusting agent is citric acid.

7. A packaged composition as claimed in claim 5, wherein the pH adjusting agent is arginine.

8. A packaged composition as claimed in claim 1, wherein the aqueous base (a) has a viscosity of 500 mPas or less at 21 s⁻¹ and 25° C.

9. A packaged composition as claimed in claim 1, wherein the propellant is selected from n-butane, iso-butane, propane and mixtures thereof.

10. A process for the preparation of a packaged composition as defined in claim 1, comprising the steps of:
(i) charging the aluminium container with the aqueous base,
(ii) sealing the container, and
(iii) adding the propellant through a valve.
